(19) United States
(12) Patent Application Publication
(10) Pub. No.: US 2024/0306694 A1
(43) Pub. Date: Sep. 19, 2024

(54) USES OF BIFIDOBACTERIUM LONGUM TRANSITIONAL MICROORGANISM

(71) Applicant: SOCIETE DES PRODUITS NESTLE S.A., Vevey (CH)

(72) Inventors: OLGA SAKWINSKA, Lausanne (CH); STEPHANE DUBOUX, St-Prex (CH); CATHERINE NGOM-BRU, Mezieres (CH); NORBERT SPRENGER, Savigny (CH)

(21) Appl. No.: 18/577,141

(22) PCT Filed: Jul. 8, 2022
(86) PCT No.: PCT/EP2022/069182

§ 371 Date: Jan. 5, 2024

Related U.S. Application Data
(60) Provisional application No. 63/220,200, filed on Jul. 9, 2021.

(51) Int. Cl. A23L 33/00; A23L 33/125; A23L 33/135

(52) U.S. Cl. CPC A23L 33/40; A23L 33/125; A23L 33/135; A23V 2400/533

(57) ABSTRACT

The present invention relates to use of a B. longum transitional microorganism that has a preferential utilization of 3-FL over 2′FL. It also relates to methods and to a composition comprising the B. longum transitional microorganism and uses thereof.

### FIELD OF THE INVENTION

The present invention relates to use of a B. longum transitional microorganism that has a preferential utilization of 3-FL over 2′FL. It also relates to methods and to a composition comprising the B. longum transitional microorganism and uses thereof.

### BACKGROUND OF THE INVENTION

Nutrition plays a critical role in the development across all areas (including cognitive, motor sensory, dentition, musculo-skeletal, immunity, and social development) in infants and young children. Further, the gastrointestinal or “gut” microbiome during infancy can play a significant role in the health and development of the infant both during infancy and later on in life (see e.g., Tanaka and Nakayama. 2017. Allergol. Int. 66(4): 515-522). Various factors, including diet, can significantly influence the microbiome structure and thus influence the health and development of an infant both during infancy and later in life. During infancy, a mammal, including a human, will transition from a diet that is composed of all or primarily a mother's milk to one of solid foods. This is referred to as the “transitional period”, “transitional feeding period”, or “weaning”. As this occurs, significant changes in the gut microbiome structure can take place due the change in diet and other stressors during that time (see e.g., Vatanen et al., 2019. Nature Microbiology. 4:470-479; Dizzell et al., 2021. PLOS ONE. https://doi.org/10.1371/journal.pone.0248924; Moore and Townsend. 2019. Open Biol. Sep; 9(9):190128; Magne et al. 2006. FEMS Microbiology Ecology, 58(3): 563-571; and Edwards C. A. Ann Nutr Metab 2017; 70:246-250). The change in microbiome structure can in turn impact the physiologic, cognitive, anatomical, health or other state or characteristic of the mammal. Although several studies have and are currently investigating the gut microbiome during infancy and young childhood, the gut microbiome and its impact on the immediate and lifelong health and well-being of the infant is far from being well characterized. Paralleling the lack of characterization and understanding of the gut microbiome in infancy and young childhood is also a paucity of compositions and formulations capable of facilitating a healthy gut microbiome appropriate for infant or young child use. As such, there exists a need for improved characterization and understanding of the gut microbiome and compositions and methods to support and/or establish a healthy gut microbiome, particularly in infants and young children.

Transition between fully milk-based diet, either breast-feeding or formula feeding, and solid foods rich in proteins and fibres results in an increase of bacterial numbers in the gut leading to the evolution in a microbial composition associated with adult individuals. Weaning is considered as a stressful and complex process and the disruption of gut microbiota can lead to microbiota dysbiosis that is linked to pathogenesis of both intestinal disorders, such as diarrhoea, IBD, IBS and coeliac disease and extra-intestinal disorders, such as allergies, asthma, metabolic syndrome, cardiovascular disease and obesity. It would be desirable to reduce the stress induced by weaning to develop and maintain a heathy gut microbiota.

As such, there exists a need for improved characterization and understanding of the gut microbiome during the weaning. Additionally, there is a need to provide nutritional compositions and methods to support the transition between milk-based diet and solid in infants and young children.

Any reference to prior art documents in this specification is not to be considered an admission that such prior art is widely known or forms part of the common general knowledge in the field.

### SUMMARY OF THE INVENTION

The inventors have found Bifidobacterium longum subsp microorganisms of a clade that is present in the gut microbiome of the transitional feeding period of mammals, particularly humans. B. longum microorganisms belonging to this clade are referred to herein as Bifidobacterium longum transitional (hereinafter B. longum transitional). B. longum transitional strains NCC 5000, NCC 5001, NCC 5002, NCC 5003 and NCC 5004 were deposited with the Institute Pasteur according to Budapest Treaty on 11^{th} of May 2021 receiving the deposit numbers CNCM 1-5683, CNCM 1-5684, CNCM 1.5685, CNCM 1-5686 and CNCM 1-5687, respectively. The inventors have shown in the U.S. provisional patent application 63/216,127 (not published yet) that the B. longum transitional microorganisms are greater in relative abundance during the transitional feeding period (e.g. weaning period) than either B. longum subsp. infantis (B. infantis) and B. longum subsp longum. Indeed, the relative abundance of B. longum subsp infantis decreases at the beginning of the transitional feeding period until the end of the transitional feeding period while B. longum subsp longum begins to increase in abundance.

The inventors have surprisingly found that the use of a B. longum transitional microorganism capable of utilizing preferentially fucosylated oligosaccharide 3-fucosyllactose (3-FL) over 2′-fucosyllactose (2′-FL) may be beneficial for the weaning period. Human milk oligosaccharides (HMOs) have been described to play different roles in numerous biological processes occurring in the human organism. Notably, HMOs are believed to positively modulate the gut microbiota. Mammalian milk contains at least 130 of these complex oligosaccharides (Urishima et al., Milk Oligosaccharides, Nova Biomedical Books, New York, ISBN: 978-1-61122-831-1). HMO composition in breast milk is complex and dynamic and the composition of HMO in breast milk varies during lactation.

In one aspect, the present invention provides a use of a Bifidobacteriumlongum transitional microorganism to promote or assist the transition from a milk-based diet to solid food in an infant and/or in a young child, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In some embodiments, the Bifidobacterium longum transitional microorganism used in the present invention may have an Average Nucleotide Identity (ANI) of of at least 96% with at least one Bifidobacterium longum strain selected in the group consisting of CNCM 1-5683, CNCM 1-5684, CNCM 1-5685, CNCM 1-5686 and CNCM 1-5687, and any combination thereof.

In some embodiments, the Bifidobacterium longum transitional microorganism used in the present invention may comprise a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

In some embodiments, the Bifidobacterium longum transitional microorganism used in the present invention may preferentially utilize 3-fucosyllactose (3-FL) over 2′-fucosyllactose (2′-FL) in a ratio between 0.1:5, preferably in a ratio between 0.1:4, more preferably in a ratio between 0.2:2.

In some embodiments, the Bifidobacterium longum transitional microorganism used in the present invention may comprise a sialidase or neuraminidase family 33 (GH33, sialidase or neuraminidase) gene having at least 60% (OPTIMAL above 80%) of identity with BLON_2348 gene present in Bifidobacterium longum subsp. infantis ATCC 15697.

In another aspect, the present invention provides a use of a Bifidobacterium longum transitional microorganism to increase short-chain fatty acids production in infant and/or in a young child, wherein the Bifidobacteriumlongum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In another aspect, the present invention provides a use of a Bifidobacterium longum transitional microorganism to reduce the presence of enteropathogens, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In another aspect, the present invention provides a use of a Bifidobacterium longum transitional microorganism to modulate the gut microbiome, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In another aspect, the present invention provides a method of promoting the growth of a Bifidobacterium longum transitional microorganism to modulate the gut microbiota of an infant and/or of a young child, the method comprising administering to the infant and/or to the young child a composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In another aspect, the present invention provides a method of promoting or assisting the transition from a milk-based diet to solid food in an infant and/or in a young child, the method comprising administering to the infant and/or to the young child a composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In another aspect, the present invention provides a composition for promoting gut microbiota adapted to metabolize both milk derived carbohydrates and fibres or any derivatives thereof, said composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In some embodiments, the composition may comprise at least one prebiotic oligosaccharide selected in the group consisting of: 2′-O-fucosyllactose (2FL), 3′-O-fucosyllactose (3FL), lactodifucotetraose/difucosyllactose (DFL), 3′-O-sialyllactose (3-SL), 6′-O-sialyllactose (6-SL) and lacto-N-tetraose (LNT) and any combination thereof.

In some embodiments, the composition may comprise at least one prebiotic oligosaccharide selected in the group consisting of 2′-O-fucosyllactose (2FL), 3′-O-fucosyllactose (3FL), lactodifucotetraose/difucosyllactose (DFL), 3′-O-sialyllactose (3-SL), 6′-O-sialyllactose (6-SL) and lacto-N-tetraose (LNT) and any combination thereof.

The composition may comprise 34 wt % to 85 wt % of 2′-FL, 10 wt % to 40 wt % of LNT, 4 wt % to 14 wt % of DFL and 9 wt % to 31 wt % of 3-SL and 6-SL combined.

In one aspect, the present invention provide a use of the composition according to the invention to promote the growth of a Bifidobacterium longum transitional microorganism that preferentially utilizes 3-fucosyllactose (3-FL) over 2′-fucosyllactose (2′-FL).

In one aspect, the present invention provides a use of the composition according to the invention to promote or assist the transition from a milk-based diet to solid food in an infant and/or in a young child.

In another aspect, the present invention provides a use of the composition according to the invention to increase short-chain fatty acids production in infant and/or in a young child.

In another aspect, the present invention provides a use of the composition according to the invention to reduce the presence of enteropathogens.

In another aspect, the present invention provides a method of promoting the growth of the Bifidobacterium longum transitional microorganism that preferentially utilizes 3-fucosyllactose (3-FL) over 2′-fucosyllactose (2′-FL) to modulate the gut microbiota of an infant and/or of a young child, the method comprising administering to the infant and/or to the young child a composition according to the invention

In another aspect, the present invention provides a method of promoting or assisting the transition from a milk-based diet to solid food in an infant and/or a young child, the method comprising administering to the infant and/or the young child a composition according to the invention.

### BRIEF DESCRIPTION OF THE DRAWINGS

FIG. 1 shows the Average Nucleotide Identity (ANI) UPGMA based phylogenetic tree of strains belonging to the B. longum species. The scale represents the percentage of identity at each branch point.

FIG. 2 is a schematic representation of the organization of the genes implicated in the degradation and the metabolization of fucosylated human milk oligosaccharides in the B. longum transitional strains, compared to B. longum subsp. infantis ATCC 15697 and B. kashiwanohense DSM 21854. Values represent percentage (%) of identity between the different genes.

FIG. 3 shows the growth of B. longum transitional strains and B. longum subsp. infantis LMG 11588 on glucose, 2′-FL or 3-FL as sole carbon source (0.5% final). Significant differences between 2′-FL and 3-FL growth for each strain were calculated using one-way ANOVA, followed by a Sidak's multiple comparison test (ns=non-significant, * p-value<0.05, ** p-value<0.01).

FIG. 4 shows the growth ratios of 3-FL over 2′-FL of B. longum transitional strains and B. longum subsp. infantis LMG 11588. FIG. 5 shows short chain fatty acids (SCFAs) production (i.e acetate, butyrate and propionate) at beginning (T0) and after 48 h (T48) of fermentation with 2-fucosylactose (2′FL). Three conditions were tested, i.e., fermentation with no supplementation, supplementation with B. longum transitional strain NCC5002, or supplementation with B. longum transitional strain NCC5004. SCFA measurements were performed by 1H-NMR technique. The Y axis corresponds to the intensity of each SCFA peak integral in arbitrary unit (au). Total SCFAs corresponds to the sum of the peak integrals of acetate, butyrate, and propionate.

FIG. 6 shows short chain fatty acids (SCFAs) production (i.e acetate, butyrate and propionate) at beginning (T0) and after 48 h (T48) of fermentation with 3-fucosylactose (3FL). Three conditions were tested, i.e., fermentation with no supplementation, supplementation with B longum transitional strain NCC5002, or supplementation with B longum transitional strain NCC5004. SCFA measurements were performed by 1H-NMR technique. The Y axis corresponds to the intensity of each SCFA peak integral in arbitrary unit (au). Total SCFAs corresponds to the sum of the peak integrals of acetate, butyrate, and propionate.

### DETAILED DESCRIPTION OF THE INVENTION

Various preferred features and embodiments of the present invention will now be described by way of non-limiting examples. The skilled person will understand that they can combine all features of the invention disclosed herein without departing from the scope of the invention as disclosed.

It must be noted that as used herein and the appended claims, the singular forms “a”, “an”, and “the” include plural referents unless the context clearly dictates otherwise.

The terms “comprising”, “comprises” and “comprised” as used therein are synonymous with “including”, “includes” or “containing”, “contains”, and are inclusive or open ended and do not exclude additional, non-recited members, elements or method steps. The terms “comprising”, “comprises” and “comprised of” also include the term “consisting of”.

Numeric ranges are inclusive of number defining the range.

All percentages are by weight unless otherwise stated.

The terms “about” or “approximatively” as used herein when referring to a measurable value such as a parameter, an amount, a temporal duration, and the like, are meant to encompass variations of and from the specific value, such as the variation of 1/−10% or less, 1/−5% or less, 1/−1% or less, and +/0.1% or less of and from the specific value, insofar such variations are appropriate to perform in the disclosed invention. It is to be understood that the value to which the modifier “about” or “approximately” refers is itself also specifically, and preferably, disclosed.

The terms “subject”, “individual” and “patient” are used interchangeably to refer to a vertebrate, preferably a mammal, more preferably a human. Mammals include but are not limited to murines, simians, humans, farm animals, sport animals and pets.

The term “infant” means a human subject under the age f 12 months or an age equivalent non-human animal.

The terms “young child” or “toddler” as used herein mean a human subject aged between 12 months and 5 years of age.

The expressions “complementary feeding period”, “complementary period”, “transitional period”, “transitional feeding period” and “weaning period” can be interchangeably used and refer to the period during which the milk, either breast milk or formula, is substituted by other foods in the diet of an infant or a young child. The infant or the young child is typically moved or transitioned gradually from exclusive milk-feeding, either breast feeding or formula feeding, to mixed diet comprising milk and/or solid foods. The transitional period depends on the infant or young child but typically falls between about 4 months and about 18 months of age, such as between about 6 and about 18 months of age, but can in some instances extend up to about 24 months or more. For humans, the weaning period typically starts between 4 and 6 months of age and is considered completed once the infant and/or the young child is no longer fed with breast milk or infant formula, typically at about 24 months of age. In some embodiments, the weaning period is between 4 and 24 months.

The expression “composition” or “nutritional composition” refer to any kind of composition or formulation that provides a nutritional benefit to an individual and that may be safely consumed by a human or an animal. Said nutritional composition may be in solid (e.g. powder), semi-solid or liquid form and may comprise one or more macronutrients, micronutrients, food additives, water, etc. For instance, the nutritional composition may comprise the following macronutrients: a source of proteins, a source of lipids, a source of carbohydrates and any combination thereof. Furthermore, the nutritional composition may comprise the following micronutrients: vitamins, minerals, fiber, phytochemicals, antioxidants, prebiotics, probiotics, and any combination thereof. The composition may also contain food additives such as stabilizers (when provided in solid form) or emulsifiers (when provided in liquid form). The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water, e.g. a reconstituted infant formula or follow-on/follow-up formula or infant cereal product or any other formulation designed for infant or young child nutrition). Generally, a nutritional composition can be formulated to be taken enterally, orally, parenterally, or intravenously, and it usually includes one of more nutrients selected from: a lipid or fat source, a protein source. and a carbohydrate source. Preferably, a nutritional composition is for oral use. In a particular embodiment, the composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The terms “fortifier” refers to liquid or solid nutritional compositions suitable for fortifying or mixing with human milk, infant formula, growing-up milk or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

An “oligosaccharide” is a saccharide polymer containing a small number (typically three to ten) of simple sugars (monosaccharides). It may refer to a carbohydrate that has greater than 2 but relatively few monosaccharide units (typically 3, 4, 5, 6, and up to 10). Exemplary oligosaccharides include, but are not limited to, fructo-oligosaccharides, galacto-oligosaccharides (raffinose, stachyose, verbascose), maltooligosaccharides, gentio-oligosaccharides, cellooligosaccharides, milk oligosaccharides (e.g., those present in secretions from mammary glands), isomalto-oligosaccharides, lactosucrose, mannooligosaccharides, melibiose-derived oligosaccharides, pectic oligosaccharides, xylo-oligosaccharides.

The term “polysaccharide” may refer to a carbohydrate that has more than ten monosaccharide units. Exemplary polysaccharides include, but are not limited to, starch, arabinogalactan, laminarin, chrysolaminarin, xylan, arabinoxylan, mannan, fucoidan and galactomannan. It is to be understood that there is not a precise cut-off or distinction between the terms oligosaccharide and polysaccharide, nor is such a distinction necessary to practice the invention.

The term “HMO” or “HMOs” refers to human milk oligosaccharide(s). These carbohydrates are highly resistant to enzymatic hydrolysis, indicating they may display essential functions not directly related to their caloric value. It has been especially illustrated they play a vital role in the early development of infants and young children, such as the maturation of the immune system. Many different kinds of HMOs are found in the human milk. Each individual oligosaccharides is based on a combination of glucose, galactose, sialic acid (N-acetylneuraminic acid), fucose and/or N-acetylglucosamine with many and varied linkages between them, thus accounting for the enormous number of different oligosaccharides in human milk—over 130 such structures have been identified so far. Almost all of them have a lactose moiety at their reducing end while sialic acid and/or fucose (when present) occupy the terminal position at the non-reducing ends. Depending on the presence of fucose and sialic acid in the oligosaccharide structure, the HMOs can be divided as non-fucosylated (neutral) or fucosylated (neutral) and sialylated (acidic) and non-sialylated molecules, respectively.

The expression “fucosylated oligosaccharide” refers to an oligosaccharide having a fucose residue. It has a neutral nature. Some examples are 2′-fucosyllactose (2-FL), 3-fucosyllactose (3-FL), difucosyllactose (DiFL), lacto-N-fucopentaose (e.g. lacto-N-fucopentaose I, lacto-N-fucopentaose II, lacto-N-fucopentaose Ill, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof. Fucosylated oligosaccharides represents the largest fraction of human milk with 2′-FL constituting up to 30% of the total HMOs. Fucosylated oligosaccharides are thought to reduce the risk of infections and inflammations and to boost growth and metabolic activity of specific commensal microbes reducing inflammatory response.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

The expressions “at least one fucosylated oligosaccharide” and “at least one N-acetylated oligosaccharide” should be understood as “at least one type of fucosylated oligosaccharide” and “at least one type of N-acetylated oligosaccharide”.

The term “sialylated oligosaccharide” refers to an oligosaccharide having a charged sialic acid residue. It has an acidic nature. Some examples are 3′-sialyllactose (3-SL) and 6′-sialyllactose (6-SL).

The term “fibers” refers to carbohydrates that are indigestible by a human or animal. Such fibers are also discussed in relation to carbohydrates herein. Suitably, the fiber can be fermented by one or more B. longum transitional microorganisms provided in the present use or composition and/or within one or more regions in the gastrointestinal tract within an organism, such as a human or non-human animal. As used herein, the expressions “fiber” or “fibers” or “dietary fiber” or “dietary fibers” within the context of the present invention indicate the indigestible portion, in small intestine, of food derived from plants which comprises two main components: soluble fiber, which dissolves in water and insoluble fiber. Mixtures of fibers are comprised within the scope of the terms above mentioned. Soluble fiber is readily fermented in the colon into gases and physiologically active byproducts and can be prebiotic and viscous. Insoluble fiber does not dissolve in water, is metabolically inert and provides bulking, or it can be prebiotic and metabolically ferment in the large intestine. Chemically, dietary fiber consists of carbohydrate polymers with three or more monomeric units which are not hydrolyzed by endogenous enzymes in the small intestine such as arabinoxylans, cellulose, and many other plant components such as resistant starch, resistant dextrins, inulin, lignin, chitins, pectins, arabinans, arabinogalactans, galactans, xylans, beta-glucans, and oligosaccharides. Non-limiting examples of dietary fibers are: prebiotic fibers such as Fructo-oligosaccharides (FOS), inulin, galacto-oligosaccharides (GOS), fruit fiber, vegetable fiber, cereal fiber, resistant starch such as high amylose corn starch.

The term “prebiotic” means non-digestible carbohydrates that beneficially affect the host by selectively stimulating the growth and/or the activity of healthy bacteria such as bifidobacterial in the colon of humans (Gibson G R, Roberfroid M B. Dietary modulation of the human colonic microbiota: introducing the concept of prebiotics. J Nutr. 1995; 125:1401-12). Mains sources ot prebiotics are plant-derived carbohydrate compounds (oligosaccahrides) and resistant starch. The prebiotic can be for example a glycan-based prebiotic, fructans such as fructooligosaccharides (FOS), inulin, galactans such as galactooligosaccharides (GOS), pectin, beta-glucans, xylooligosaccharides

The term “metabolize” is used herein to mean that a substrate can by broken down, adsorbed and/or utilized by a microorganism. For example, the substrate may promote and/or contribute to the growth and/or survival of the microorganism. Growth and/or survival of the B. longum transitional strain may be determined by measuring the abundance of 16S rDNA—for example using PCR methods.

The expression “capable of utilizing prebiotic oligosaccharides” may mean that the Bifidobacterium longum transitional microorganism encodes at least one CAZyme which is capable of metabolizing the prebiotic oligosaccharide substrate. The expression “capable of utilizing prebiotic oligosaccharides” may mean that the prebiotic oligosaccharide substrate is capable of promoting growth and/or survival of the Bifidobacterium longum transitional microorganism. Growth and/or survival of the B. longum transitional strain may be determined by measuring the abundance of 16S rDNA—for example using PCR methods.

The term “probiotic” means microbial cell preparation or components of microbial cells with a beneficial effect on the health or wellbeing of the host (Salminen S, Ouwehand A. Benno Y. et al. “Probiotics: how should they be defined” Trends Food Sci. Technol. 1999:10 107-10). The microbial cells are generally bacteria or yeasts.

The term “cfu” should be understood as colony forming unit.

The “gut microbiota” is the composition of microorganisms (including bacteria, archaea and fungi) that live in the digestive tract.

The term “gut microbiome” may encompass both the “gut microbiota” and their “theater of activity”, which may include their structural elements (nucleic acid, proteins, lipids, polysaccharides), metabolites (signaling molecules, toxins, organic and inorganic molecules) and molecules produced by coexisting hosts and structured by the surrounding environmental conditions (Berg, G., et al., 2020. Microbiome, 8(1), pp. 1-22).

In the present invention, the term “gut microbiome” can be used interchangeably with the term “gut microbiota”.

The term “SCFA” means short chain fatty acid(s). SCFAs are the products of colonic bacterial degradation of unabsorbed starch and non-strach polysaccharides (fiber).

Bifidobacterium longum Transitional Microorganism

The inventors have found a Bifidobacterium longum transitional microorganism capable of utilizing prebiotic oligosaccharides and characterized in that it utilizes preferentially 3-fucosyllactose (3-FL) over 2′-fucosyllactose (2′-FL). The expressions “B. Longum transitional microorganism”, “Bifidobacterium Longum transitional microorganism”, “B. Longum transitional strain” and “Bifidobacterium Longum transitional strain” are used interchangeably in the present invention.

In some embodiments, a Bifidobacterium longum transitional microorganism has an Average Nucleotide Identity (ANI) of at least 96% with at least one Bifidobacterium longum strain selected in the group consisting of CNCM 1-5683, CNCM 1-5684, CNCM 1-5685, CNCM 1-5686 and CNCM 1-5687, and any combination thereof. In some embodiments, a Bifidobacterium longum transitional microorganism has an ANI of about 96% to 100% with at least one Bifidobacterium longum strain selected in the group consisting of CNCM 1-5683, CNCM 1-5684, CNCM 1-5685, CNCM 1-5686 and CNCM 1-5687, and any combination thereof. In some embodiments, a Bifidobacterium longum transitional microorganism has an ANI of about 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.6%, 98.7%, 98.8%, 98.9%, 99%, 99.1%, 99.2%, 99.3%, 99.4%, 99.5%, 99.6%, 99.7%, 99.8%, 99.9%, or 100% with at least one Bifidobacterium longum strain selected in the group consisting of CNCM I-5683, CNCM 1-5684, CNCM 1-5685, CNCM 1-5686 and CNCM 1-5687, and any combination thereof. In some embodiments, a Bifidobacterium longum transitional microorganism has an ANI of at least 96%, of at least 96.1%, of at least 96.2%, of at least 96.3%, of at least 96.4%, of at least 96.5%, of at least 96.6%, of at least 96.7%, of at least 96.8%, of at least 96.9%, of at least 97%, of at least 97.1%, of at least 97.2%, of at least 97.3%, of at least 97.4%, of at least 97.5%, of at least 97.6%, of at least 97.7%, of at least 97.8%, of at least 97.9%, of at least 98%, of at least 98.1%, of at least 98.2%, of at least 98.3%, of at least 98.4%, of at least 98.5%, of at least 98.6%, of at least 98.6%, of at least 98.7%, of at least 98.8%, of at least 98.9%, of at least 99%, of at least 99.1%, of at least 99.2%, of at least 99.3%, of at least 99.4%, of at least 99.5%, of at least 99.6%, of at least 99.7%, of at least 99.8%, of at least 99.9% with at least one Bifidobacterium longum strain selected in the group consisting of CNCM 1-5683, CNCM 1-5684, CNCM 1-5685, CNCM 1-5686 and CNCM I-5687, and any combination thereof.

The “Average Nucleotide Identity (ANI)” is a term of art that refers to a distance based approach to delineate species based on pair-wise comparisons of their genome sequences and is an in silico alternative to the traditional DNA-DNA hybridization (DDH) techniques that have been used for phylogenetic definition of a species (Goris et al., 2007, “DNA-DNA hybridization values and their relationship to whole-genome sequence similarities”, Int. J. Syst. Evol. Microbiol. 57: 81-91). Based on DDH, strains with greater than 70% relatedness would be considered to belong to the same species (see e.g., Wayne et al., 1987, Report of the Ad-Hoc-Committee on Reconciliation of Approaches to Bacterial Systematics. Int J Syst Bacteriol 37: 463-464). ANI is similar to the aforementioned 70% DDH cutoff value and can be used for species delineation. ANI has been evaluated in multiple labs and has become the gold standard for species delineation (see e.g., Kim et al., 2014, “Towards a taxonomic coherence between average nucleotide identity and 16S rRNA gene sequence similarity for species demarcation of prokaryotes”, Int. J. Syst. Evol. Micr. 64: 346-351; Richter et al., 2009, “Shifting the genomic gold standard for the prokaryotic species definition”, P Natl Acad Sci USA 106: 19126-19131; and Chan et al., 2012, “Defining bacterial species in the genomic era: insights from the genus Acinetobacter”, Bmc. Microbiol. 12)).

The ANI of the shared genes between two strains is known to be a robust means to compare genetic relatedness among strains, and that ANI values of about 95% correspond to the 70% DNA-DNA hybridization standard for defining a species. See, e.g., Konstantinidis and Tiedje, Proc Natl Acad Sci USA, 102(7):2567-72 (2005); and Goris et al., Int Syst Evol Microbiol. 57(Pt 1):81-91 (2007). The ANI between two bacterial genomes is calculated from pair-wise comparisons of all sequences shared between any two strains and can be determined, for example, using any of a number of publicly available ANI tools, including but not limited to OrthoANI with usearch (Yoon et al. Antonie van Leeuwenhoek 110:1281-1286 (2017)); ANI Calculator, JSpecies (Richter and Rossello-Mora, Proc Natl Acad Sci USA 106:19126-19131 (2009)); and JSpeciesWS (Richter et al., Bioinformatics 32:929-931 (2016)). Other methods for determining the ANI of two genomes are known in the art. See, e.g., Konstantinidis, K. T. and Tiedje, J. M., Proc. Natl. Acad. Sci. U.S.A., 102: 2567-2572 (2005); and Varghese et al., Nucleic Acids Research, 43(14):6761-6771 (2015). In a particular embodiment, the ANI between two bacterial genomes can be determined, for example, by averaging the nucleotide identity of orthologous genes identified as bidirectional best hits (BBHs). Protein-coding genes of a first genome (Genome A) and second genome (Genome B) are compared at the nucleotide level using a similarity search tool, for example, NSimScan (Novichkov et al., Bioinformatics 32(15): 2380-23811 (2016). The results are then filtered to retain only the BBHs that display at least 70% sequence identity over at least 70% of the length of the shorter sequence in each BBH pair. The ANI of Genome A to Genome B is defined as the sum of the percent identity times the alignment length for all BBHs, divided by the sum of the lengths of the BBH genes. These and ANI determination techniques are known in the art and are described elsewhere herein.

In the present embodiment, a Bifidobacterium longum microorganism selected from the group consisting of CNCM 1-5683, CNCM 1-5684, CNCM 1-5685, CNCM 1-5686 and CNCM 1-5687, represents the reference genome to which a microbial genome is compared. Genome sequences for B. longum transitional strains NCC 5000 (CNCM I-5683), NCC 5001 (CNCM 1-5684), NCC 5002 (CNCM 1.5685), NCC 5003 (CNCM 1-5686) and NCC 5004 (CNCM 1-5687) are available via Joint Genome Project (JGI) Study number: Gs0156595 (https://genome.jgi.doe.gov/portal/). Analysis project numbers for each genome are as follows:

| | |
|---|---|
| B. longum NCC 5000 | Ga0527908 |
| B. longum NCC 5001 | Ga0529016 |
| B. longum NCC 5002 | Ga0529017 |
| B. longum NCC 5003 | Ga0529018 |
| B. longum NCC 5004 | Ga0529019 |

Carbohydrate-active enyzymes (CAZymes) are responsible for the synthesis and breakdown of glycoconjugates, oligo- and polysaccharides. They typically correspond to 1-5% of the genes in the living organism. Glycoconjugates, oligo- and polysaccharides play essential roles in many biological functions, for example as structure and energy reserve components and in many intra- and intercellular events. CAZymes are often involved in immune and host-pathogens interactions and are implicated in some human diseases. The Carbohydrate Active Enzyme (CAZy) classification is a sequence-based family classification system that correlate with the structure and molecular mechanism of CAZymes. The CAZy classification is widely used by the scientific community. Suitably, the Bifidobacterium longum transitional microorganism of the present invention advantageously is capable of utilizing HMOs. HMO is capable of promoting growth and/or survival of the Bifidobacterium longum transitional microorganism (e.g. when added to an anaerobic culture of the Bifidobacterium longum transitional microorganism). Growth and/or survival of the Bifidobacterium longum transitional microorganism may be determined by measuring the abundance of 16S rDNA—for example using PCR methods. Suitably, Bifidobacterium longum transitional microorganism of the present invention advantageously harbors genes encoding for CAZymes allowing the utilization of fucosylated oligosaccharides. This allows effective utilization of the fucosylated oligosaccharides that are highly present in the breast milk at weaning and hence can participate to an appropriate development of the gut microbiome of an infant and/or a young child.

In some embodiments, a Bifidobacterium longum transitional microorganism comprises a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

In some embodiments, a Bifidobacterium longum transitional microorganism comprises a glycosyl hydrolase family 95 (GH95, α-L-galactosidase; α-L-fucosidase; α-1,2-L-fucosidase) gene having about 60%, about 61%, about 62%, about 63%, about 64%, about 65%, about 66%, about 67%, about 68%, about 69%, about 70%, about 71%, about 72%, about 74%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having about 61%, about 62%, about 63%, about 64%, about 65%, about 66%, about 67%, about 68%, about 69%, about 70%, about 71%, about 72%, about 74%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% identity with BLON_2336 gene present in Bifidobacterium longum subsp. infantis ATCC 15697.

In some embodiments, a Bifidobacterium longum transitional microorganism comprises a glycosyl hydrolase family 95 (GH95, α-L-galactosidase; α-L-fucosidase; α-1,2-L-fucosidase) gene having at least 60%, at least 61%, at least 62%, at least 63%, at least 64%, at least 65%, at least 66%, at least 67%, at least 68%, at least 69%, at least 70%, at least 71%, at least 72%, at least 74%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60%, at least 61%, at least 62%, at least 63%, at least 64%, at least 65%, at least 66%, at least 67%, at least 68%, at least 69%, at least 70%, at least 71%, at least 72%, at least 74%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with BLON_2336 gene present in Bifidobacterium longum subsp. infantis ATCC 15697.

The Bifidobacterium longum transitional microorganism of the present invention advantageously harbors genes encoding for CAZymes allowing cleavage of sialic residues from glycans such as sialylated oligosachharides, glycoproteins and glycolipids. This allows effective utilization of the sialylated oligosaccharides that are present in the breast milk at weaning and hence can participate to an appropriate development of the gut microbiome of an infant and/or a young child. It may also help in preventing presence of enteropathogens.

In some embodiments, a Bifidobacterium longum transitional microorganism comprises a sialidase or neuraminidase family 33 (GH33, sialidase or neuraminidase) gene having at least 60% identity with BLON_2348 gene present in Bifidobacterium longum subsp. infantis ATCC 15697.

In some embodiments, a Bifidobacterium longum transitional microorganism comprises sialidase or neuraminidase family 33 (GH33, sialidase or neuraminidase) gene having about 60%, about 61%, about 62%, about 63%, about 64%, about 65%, about 66%, about 67%, about 68%, about 69%, about 70%, about 71%, about 72%, about 74%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% identity with BLON_2348 gene present in Bifidobacterium longum subsp. infantis ATCC 15697.

In some embodiments, In some embodiments, a Bifidobacterium longum transitional microorganism comprises sialidase or neuraminidase family 33 (GH33, sialidase or neuraminidase) gene having at least 60%, at least 61%, at least 62%, at least 63%, at least 64%, at least 65%, at least 66%, at least 67%, at least 68%, at least 69%, at least 70%, at least 71%, at least 72%, at least 74%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with BLON_2348 gene present in Bifidobacterium longum subsp. infantis ATCC 15697.

In some embodiments, the Bifidobacterium longum transitional microorganism of the present invention is isolated from human.

In some other embodiments, the Bifidobacterium longum transitional microorganism is not of the subspecies Bifidobacterium longum subsp. longum or Bifidobacterium longum subsp. infantis.

Uses of a Bifidobacterium longum Transitional Microorganism

The inventors have found that the Bifidobacterium longum transitional microorganism of the present invention may be capable of promoting or assisting the transition from a milk-based diet to solid food in an infant and/or in a young child. Indeed, as described in U.S. provisional patent application 63/216,127 (not published yet), the Bifidobacterium longum transitional microorganism appears to be of greater abundance than either B. longum subsp. infantis (B. infantis) and B. longum subsp. longum. In addition, the inventors have found that Bifidobacterium longum transitional microorganism is capable of utilizing preferentially 3-FL over 2′-FL. It has been shown that 3-FL is the human milk oligosaccharide that shows the greatest increase in the human breast milk during the period of transition between milk-based diet and solid food. Without to be bound by the theory, the inventors think that the B. longum transitional strains are particularly adapted to support the weaning period as they preferentially utilize 3-FL over 2′-FL.

Consequently, the present invention relates in part to the use of a Bifidobacterium longum transitional microorganism to promote or assist the transition from a milk-based diet to solid food in an infant and/or in a young child, wherein the Bifidobacteriumlongum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

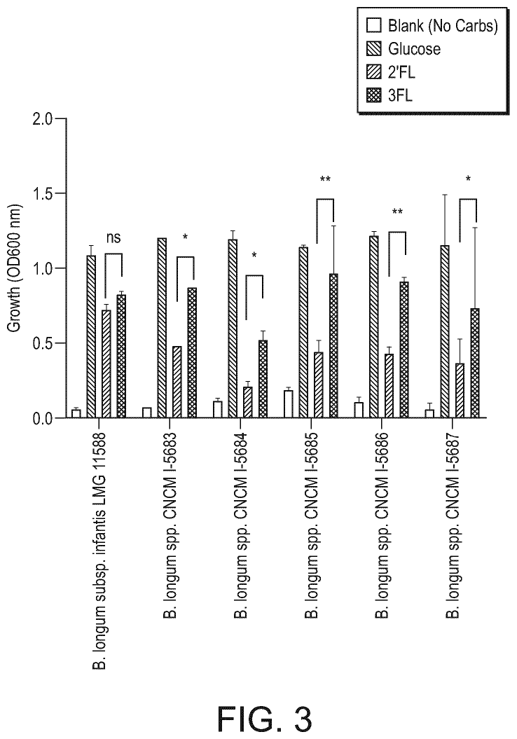

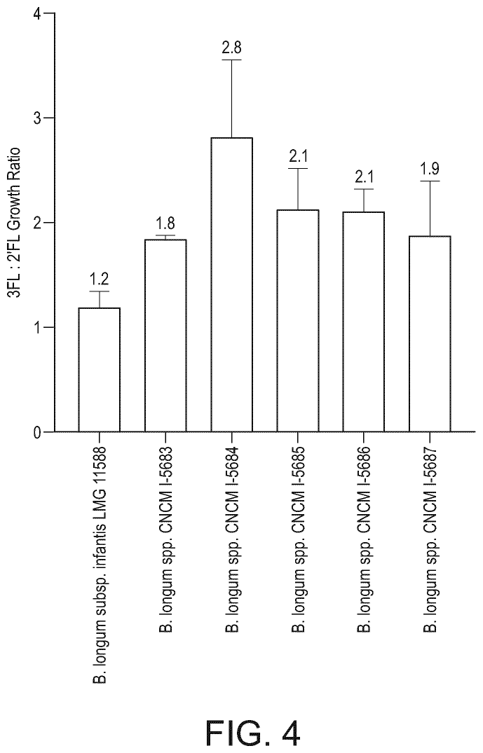

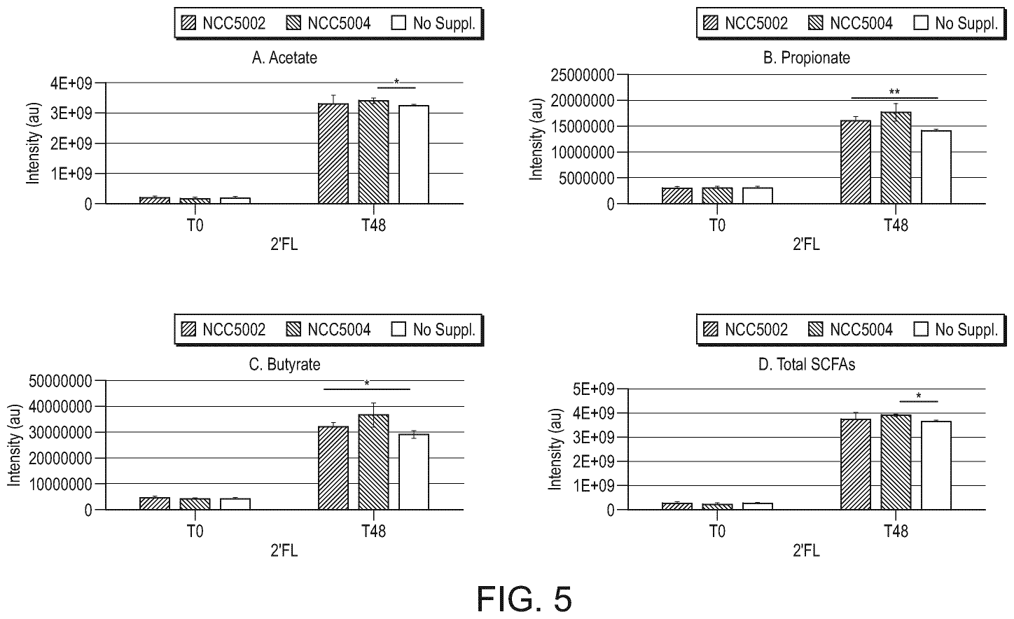

In some embodiments, the Bifidobacterium longum transitional microorganism used in the present invention utilizes more efficiently 3-FL than 2′-FL, as demonstrated by a better growth in example 2 and FIGS. 3 and 4.

The composition of HMO in breast milk varies during lactation. B. longum transitional strains harbors genes encoding for CAZymes capable of utilizing fucosylated HMOs (see example 1). The inventors surprisingly have found that the B. longum transitional microorganism according to the present invention grows better on 3-FL than on 2′-FL (see example 2 and FIGS. 3 and 4), which indicates a preferential or a more efficient utilization of 3-FL over 2′-FL by the B. longum transitional strains. Without to be bound by the theory, it is believed that this preferential utilization of 3-FL over 2′-FL can promote or assist the weaning from milk-based diet to solid food diet in infants and/or young children through efficient utilization of the HMO found in breast milk during this period. Suitably, B. longum transitional strains may produce metabolites that would be beneficial for the development of a diverse and healthy gut microbiome. For example, B. longum transitional may produce acetate, a metabolite that is the preferential substrate for several butyrate-producing microorganisms, hence favoring the development of butyrate-producing microorganisms which in turn may have a beneficial effect on immune development. The presence of HMO and of the B. longum transitional strain would increase acetate levels in the gut which in turn would promote the appearance of several other beneficial microbes, hence participating to the development of a diverse and healthy gut microbiota during the weaning period.

In some embodiments, the Bifidobacterium longum transitional microorganism used according to the present invention has an ANI of at least 96% with at least one with at least one Bifidobacterium longum strain selected in the group consisting of CNCM 1-5683, CNCM 1-5684, CNCM 1-5685, CNCM 1-5686 and CNCM 1-5687, and any combination thereof.

In some embodiments, the Bifidobacterium longum transitional microorganism used according to the present invention comprises a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

In some embodiments, the Bifidobacterium longum transitional microorganism used according to the present invention comprises a sialidase or neuraminidase family 33 (GH33, sialidase or neuraminidase) gene having at least 60% identity with BLON_2348 gene present in Bifidobacterium longum subsp. infantis ATCC 15697.

In some embodiments, the 3-fucosyllactose (3-FL) is preferentially utilized by the Bifidobacterium longum transitional microorganism over 2′-fucosyllactose (2′-FL) in a ratio between 0.1:5, preferably in a ratio between 0.1:4, more preferably in a ratio between 0.2:2. This preferential or more efficient utilization of 3-FL over 2′-FL can promote or assist the weaning from milk-based diet to solid food diet in infants and/or young children through efficient utilization of the HMO found in breast milk during this period.

Another aspect of the present invention provides the use of a Bifidobacterium longum transitional microorganism to increase the short-chain fatty acids (SCFA) production in an infant and/or in a young child, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

The expressions “to increase the short-chain fatty acids (SCFA) production” or “increasing the short chain fatty acids (SCFA) production” mean that the amount of systemic and/or colonic SCFA is higher in an individual fed with the nutritional composition according to the present invention in comparison with a standard. The SCFA production may be measured by techniques known by the skilled person such as Gas-Liquid Chromatography. SCFA may be produced either by the Bifidobacterium longum transitional strains or by other members of the microbiota. Examples of SCFA are acetate, butyrate, propionate and lactate. Those metabolites have advantageous impacts or effects cross-feeding other members of the microbiota, for example acetate is the substrate used by butyrate-producer microorganisms. SCFA also has an advantageous direct effect on improving the permeability of gut barrier and on promoting immune development.

Other embodiments of the present invention provides the use of a Bifidobacterium longum transitional microorganism to increase the short-chain fatty acids (SCFA) production in an infant and/or in a young child wherein the Bifidobacterium longum microorganism has an Average Nucleotide Identity (ANI) of at least 96% with at least one Bifidobacterium longum strain selected in the group consisting of CNCM 1-5683, CNCM 1-5684, CNCM 1-5685, CNCM 1-5686 and CNCM 1-5687, and any combination thereof.

Other embodiments of the present invention relate to the use of a Bifidobacterium longum transitional microorganism to increase the short-chain fatty acids (SCFA) production in an infant and/or in a young child wherein the Bifidobacterium longum transitional microorganism comprises a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

Other embodiments of the present invention relate to the use of a Bifidobacterium longum transitional microorganism to increase the short-chain fatty acids (SCFA) production in an infant and/or in a young child wherein the Bifidobacterium longum transitional microorganism comprises a sialidase or neuraminidase family 33 (GH33, sialidase or neuraminidase) gene having at least 60% identity with BLON_2348 gene present in Bifidobacterium longum subsp. infantis ATCC 15697.

Other embodiments of the present invention relate to the use of a Bifidobacterium longum transitional microorganism to increase the short-chain fatty acids (SCFA) production in an infant and/or in a young child wherein 3-fucosyllactose (3-FL) is preferentially utilized by the Bifidobacterium longum transitional microorganism over 2′-fucosyllactose (2′-FL) in a ratio between 0.1:5, preferably in a ratio between 0.1:4, more preferably in a ratio between 0.2:2.

Another aspect of the present invention provides the use of a Bifidobacterium longum transitional microorganism to reduce the presence of enteropathogens in the gut of an infant and/or a young child, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

The term “enteropathogen” refers to any organism that causes disease of the intestinal tract.

As previously mentioned, the inventors have found that Bifidobacterium longum transitional microorganism is capable of utilizing preferentially 3-FL over 2′-FL. It has been shown that 3-FL is the human milk oligosaccharide that shows the greatest increase in the human breast milk during the period of transition between milk-based diet and solid food. The presence of HMO and of the B. longum transitional strain would increase acetate and other SCFA levels in the gut which in turn results a reduction of the pH. It is known that survival of enteropathogen is impaired at low pH. Furthermore, the Bifidobacteriumlongum transitional strains may produce other anti-bacterial substances, such as bactriocin or other small peptides, that would limit the growth of enteropathogens. Therefore, Bifidobacterium longum transitional microorganism is capable of utilizing preferentially 3-FL over 2′-FL may advantageously reduce the presence of enteropathogens in the gut of an infant and/or a young child.

Some embodiment relates the use of a Bifidobacterium longum transitional microorganism to reduce the presence of enteropathogens in the gut of an infant and/or a young child, wherein the Bifidobacterium longum microorganism has an Average Nucleotide Identity (ANI) of at least 96% with at least one Bifidobacterium longum strain selected in the group consisting of CNCM 1-5683, CNCM 1-5684, CNCM 1-5685, CNCM 1-5686 and CNCM 1-5687, and any combination thereof.

Some embodiments relate the use of a Bifidobacterium longum transitional microorganism to reduce the presence of enteropathogens in the gut of an infant and/or a young child wherein the Bifidobacterium longum transitional microorganism comprises a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

Some embodiments relate the use of a Bifidobacterium longum transitional microorganism to reduce the presence of enteropathogens in the gut of an infant and/or a young child wherein the Bifidobacterium longum transitional microorganism comprises a sialidase or neuraminidase family 33 (GH33, sialidase or neuraminidase) gene having at least 60% identity with BLON_2348 gene present in Bifidobacterium longum subsp. infantis ATCC 15697.

Some embodiments relate the use of a Bifidobacterium longum transitional microorganism to reduce the presence of enteropathogens in the gut of an infant and/or a young child wherein 3-fucosyllactose (3-FL) is preferentially utilized by the Bifidobacterium longum transitional microorganism over 2′-fucosyllactose (2′-FL) in a ratio between 0.1:5, preferably in a ratio between 0.1:4, more preferably in a ratio between 0.2:2.

Another aspect of the present invention provides the use of a Bifidobacterium longum transitional microorganism to modulate the gut microbiome wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

The presence of HMO and of the B. longum transitional strain would promote either the reduction or the increase of nutrients, metabolites and/or other microorganisms, thereby modulating the gut microbiome.

Some embodiments relate the use of a Bifidobacterium longum transitional microorganism to modulate the gut microbiome, wherein the Bifidobacterium longum microorganism has an Average Nucleotide Identity (ANI) of at least 96% with at least one Bifidobacterium longum strain selected in the group consisting of CNCM 1-5683, CNCM 1-5684, CNCM 1-5685, CNCM 1-5686 and CNCM I-5687, and any combination thereof.

Some embodiments relate the use of a Bifidobacterium longum transitional microorganism to modulate the gut microbiome, wherein the Bifidobacterium longum transitional microorganism comprises a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

Some embodiments relate the use of a Bifidobacterium longum transitional microorganism to modulate the gut microbiome, wherein the Bifidobacterium longum transitional microorganism comprises a sialidase or neuraminidase family 33 (GH33, sialidase or neuraminidase) gene having at least 60% identity with BLON_2348 gene present in Bifidobacterium longum subsp. infantis ATCC 15697.

Some embodiments relate the use of a Bifidobacterium longum transitional microorganism to modulate the gut microbiome, wherein 3-fucosyllactose (3-FL) is preferentially utilized by the Bifidobacterium longum transitional microorganism over 2′-fucosyllactose (2′-FL) in a ratio between 0.1:5, preferably in a ratio between 0.1:4, more preferably in a ratio between 0.2:2.

### Methods

One aspect of the present invention provides a method of promoting the growth of a Bifidobacterium longum transitional microorganism to modulate the gut microbiota of an infant and/or of a young child, the method comprising administering to the infant and/or to the young child a composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

Another aspect of the present invention provides a method of promoting/assisting the transition from a milk-based diet to solid food in an infant and/or in a young child, the method comprising administering to the infant and/or to the young child a composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

In some embodiments, the Bifidobacterium longum transitional has an Average Nucleotide Identity (ANI) of at least 96% with at least one Bifidobacterium longum strain selected in the group consisting of CNCM 1-5683, CNCM 1-5684, CNCM 1-5685, CNCM 1-5686 and CNCM 1-5687, and any combination thereof.

In some embodiments, the Bifidobacterium longum transitional comprises a glycosyl hydrolase family 95 (GH95, α-L-galactosidase; α-L-fucosidase; α-1,2-L-fucosidase) gene having at least 60% of identity with BLON_2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

In some embodiments, the Bifidobacterium longum transitional comprises a sialidase or neuraminidase family 33 (GH33, sialidase or neuraminidase) gene having at least 60% identity with BLON_2348 gene present in Bifidobacterium longum subsp. infantis ATCC 15697.

In some embodiments, 3-fucosyllactose (3-FL) is preferentially utilized by the Bifidobacterium longum transitional microorganism over 2′-fucosyllactose (2′-FL) in a ratio between 0.1:5, preferably in a ratio between 0.1:4, more preferably in a ratio between 0.2:2.

The Bifidobacterium longum transitional microorganism can be included in the composition in an amount from about 10³ to 10¹² cfu of probiotic strain, more preferably between 10⁷ and 10¹² cfu such as between 10⁸ and i0¹⁰ cfu of probiotic strain per g of composition on a dry weight basis. In one embodiment, the Bifidobacterium longum transitional microorganism is viable. In another embodiment the Bifidobacterium longum transitional microorganism is non-replicating or inactivated. There may be both viable and inactivated Bifidobacterium longum transitional microorganisms in some other embodiments.

In some embodiments, the composition comprises at least one prebiotic oligosaccharide selected in the group consisting of 2′-O-fucosyllactose (2FL), 3′-O-fucosyllactose (3FL), lactodifucotetraose/difucosyllactose (DFL), 3′-O-sialyllactose (3′-SL), 6′-O-sialyllactose (6′-SL) and lacto-N-tetraose (LNT) and any combination thereof.

In some embodiments, the composition comprises
26 wt % to 65 wt % of 2′-FL, preferably 32 wt % to 54 wt %;
10 wt % to 40 wt % of LNT, preferably 11 wt % to 20 wt %;
4 wt % to 14 wt % of DFL, preferably 4 wt % to 8 wt %;
9 wt % to 31 wt % of 3′-SL and 6′-SL combined, preferably 8 wt % to 22 wt %; and
12 wt % to 38 wt % of 3-FL, preferably 17 wt % to 31 wt %.

The composition may comprise between 0.001 g/L to 12 g/L of 2′-FL, preferably between 0.002 g/L to 10 g/L of 2′-FL, more preferably between 0.005 g/L to 5 g/L of 2′-FL.

The composition may comprise between 0.001 g/L to 5 g/L of DFL, preferably between 0.002 g/L to 4 g/L of DFL, more preferably between 4 g/L to 3 g/L of DFL.

The composition may comprise between 0.01 g/L to 6 g/L of LNT, preferably between 0.025 g/L to 5 g/L of LNT, more preferably between 0.05 g/L to 1 g/L of LNT.

The composition may comprise between 0.001 g/L to 2 g/L of 6′-SL, preferably between 0.002 g/L to 1.5 g/L of 6′-SL, more preferably between 0.005 g/L to 1 g/L of 6′-SL.

The composition may comprise between 0.01 g/L to 2 g/L of 3′-SL, preferably between 0.025 g/L to 1.5 g/L of 3′-SL, more preferably between 0.05 g/L to 1 g/L of 3′-SL.

The composition may comprise between 0.01 g/L to 7 g/L of 3-FL, preferably between 0.025 g/L to 6 g/L of 3-FL, more preferably between 0.05 g/L to 5 g/L of 3-FL.

In some embodiments, the composition comprises Bifidobacterium longum transitional preferentially utilizing 3-fucosyllactose (3-FL) over 2′-fucosyllactose (2′-FL) mixed with 3′-O-fucosyllactose (3-FL) and lacto-N-tetraose (LNT). The composition may comprise between 10³ to 10¹² cfu of probiotic strain, more preferably between 107 and 10¹² cfu such as between 10⁸ and 10¹⁰ cfu of probiotic strain per g of composition on a dry weight basis mixed with 3-FI in an amount between 0.01 g/L to 7 g/L of 3-FL, preferably between 0.025 g/L to 6 g/L of 3-FL, more preferably between 0.05 g/L to 5 g/L of 3-FL and with LNT in an amount between 0.01 g/L to 6 g/L of LNT, preferably between 0.025 g/L to 5 g/L of LNT, more preferably between 0.05 g/L to 1 g/L of LNT.

Suitably, the 3′-O-fucosyllactose (3′FL) and lacto-N-tetraose (LNT) comprised in the composition promote the growth of a Bifidobacterium longum transitional microorganism that preferentially utilizes 3-fucosyllactose (3-FL) over 2′-fucosyllactose (2′-FL).

### Compositions

One aspect of the present invention provides a composition for promoting gut microbiota adapted to metabolize both milk derived carbohydrates and fibres or any derivatives thereof, said composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

The composition may suitably be administered to an individual, for example an infant or a young child, in any suitable form such as a nutritional composition in a dosage unit (for example a tablet, a capsule, a sachet of powder, etc). the composition may be in powder, semi-liquid or liquid form. The composition may be added to a nutritional composition, an infant formula, a food composition, a supplement for infant or young child, a baby food, a follow-up formula, a growing-up milk, an infant cereal or a fortifier. In some embodiments, the composition of the present invention is an infant formula, a baby food, an infant cereal, a growing-up milk, a supplement or fortifier that may be intended for infants or young child.

By way of example, the composition may comprise further components which may be beneficial during the weaning period.

The Bifidobacterium longum transitional microorganism can be included in the composition in an amount from about 10³ to 10¹² cfu of probiotic strain, more preferably between 107 and 10¹² cfu such as between 10⁸ and 10¹⁰ cfu of probiotic strain per g of composition on a dry weight basis. In one embodiment, the Bifidobacterium longum transitional microorganism is viable. In another embodiment the Bifidobacterium longum transitional microorganism is non-replicating or inactivated. There may be both viable and inactivated Bifidobacterium longum transitional microorganisms in some other embodiments.

In some embodiments, the composition comprises at least one prebiotic oligosaccharide selected in the group consisting of 2′-O-fucosyllactose (2FL), 3′-O-fucosyllactose (3FL), lactodifucotetraose/difucosyllactose (DFL), 3′-O-sialyllactose (3′-SL), 6′-O-sialyllactose (6′-SL) and lacto-N-tetraose (LNT) and any combination thereof.

In some embodiments, the composition comprises
26 wt % to 65 wt % of 2′-FL, preferably 32 wt % to 54 wt %;
10 wt % to 40 wt % of LNT, preferably 11 wt % to 20 wt %;
4 wt % to 14 wt % of DFL, preferably 4 wt % to 8 wt %;
9 wt % to 31 wt % of 3′-SL and 6′-SL combined, preferably 8 wt % to 22 wt %; and
12 wt % to 38 wt % of 3-FL, preferably 17 wt % to 31 wt %.

The composition may comprise between 0.001 g/L to 12 g/L of 2′-FL, preferably between 0.002 g/L to 10 g/L of 2′-FL, more preferably between 0.005 g/L to 5 g/L of 2′-FL.

The composition may comprise between 0.001 g/L to 5 g/L of DFL, preferably between 0.002 g/L to 4 g/L of DFL, more preferably between 4 g/L to 3 g/L of DFL.

The composition may comprise between 0.01 g/L to 6 g/L of LNT, preferably between 0.025 g/L to 5 g/L of LNT, more preferably between 0.05 g/L to 1 g/L of LNT.

The composition may comprise between 0.001 g/L to 2 g/L of 6′-SL, preferably between 0.002 g/L to 1.5 g/L of 6′-SL, more preferably between 0.005 g/L to 1 g/L of 6′-SL.

The composition may comprise between 0.01 g/L to 2 g/L of 3′-SL, preferably between 0.025 g/L to 1.5 g/L of 3′-SL, more preferably between 0.05 g/L to 1 g/L of 3′-SL.

The composition may comprise between 0.01 g/L to 7 g/L of 3-FL, preferably between 0.025 g/L to 6 g/L of 3-FL, more preferably between 0.05 g/L to 5 g/L of 3-FL.

In some embodiments, the composition comprises Bifidobacterium longum transitional microorganism preferentially utilizing 3-fucosyllactose (3-FL) over 2′-fucosyllactose (2′-FL) mixed with 3′-O-fucosyllactose (3-FL) and lacto-N-tetraose (LNT). The composition may comprise between 10³ to 10¹² cfu of probiotic strain, more preferably between 107 and 10¹² cfu such as between 10⁸ and 10¹⁰ cfu of probiotic strain per g of composition on a dry weight basis mixed with 3-FI in an amount between 0.01 g/L to 7 g/L of 3-FL, preferably between 0.025 g/L to 6 g/L of 3-FL, more preferably between 0.05 g/L to 5 g/L of 3-FL and with LNT in an amount between 0.01 g/L to 6 g/L of LNT, preferably between 0.025 g/L to 5 g/L of LNT, more preferably between 0.05 g/L to 1 g/L of LNT.

Suitably, the 3′-O-fucosyllactose (3′FL) and lacto-N-tetraose (LNT) comprised in the composition promote the growth of a Bifidobacterium longum transitional microorganism that preferentially utilizes 3-fucosyllactose (3-FL) over 2′-fucosyllactose (2′-FL).

### Use of the Composition and Methods

One aspect of the present invention provides the use of a composition according to the invention to promote the growth of a Bifidobacterium longum microorganism that preferentially utilizes 3-fucosyllactose (3-FL) over 2′-fucosyllactose (2′-FL) to modulate the gut microbiota of an infant and/or of a young child.

Another aspect of the present invention provides the use of a composition according to the invention to promote/assist the transition from a milk-based diet to solid food in an infant and/or a young child.

Another aspect of the present invention provides the use of a composition according to the invention to increase the levels of short-chain fatty acid to modulate the gut microbiota of an infant and/or a young child.

Another aspect of the present invention provides the use of a composition according to the invention to increase the short-chain fatty acids (SCFA) production in an infant and/or in a young child. SCFAs play an important role in the maintenance of gut and metabolic health. The three main SCFAs, namely acetate, propionate and butyrate may be key mediators of the beneficial effects elicited by the gut microbiome. Microbial SCFA production is essential for the gut integrity by regulating the luminal pH, mucus production, providing fuel for epithelial cells and effects on immune function. Therefore, increasing SCFA production may have the advantage of improving gut and metabolic health.

Suitably, the present invention increases production of acetate, propionate, and/or butyrate and any combination thereof.

Another aspect of the present invention provides the use of a composition according to the invention to reduce the presence of enteropathogens in the gut of an infant and/or a young child. The reduction of entheropathogens in the gut may be the result of increased SCFA production that may results in improved gut barrier and gut functions. For example, the use of the composition according to the present invention may increase the gut epithelial barrier resistance. This could be advantageous to prevent the entry of pathogenic microorganisms such as enteropathogens.

Another aspect of the present invention provides a method of promoting the growth of the Bifidobacterium longum transitional microorganism that preferentially utilizes 3-fucosyllactose (3-FL) over 2′-fucosyllactose (2′-FL) to modulate the gut microbiota of an infant and/or of a young child, the method comprising administering to the infant and/or to the young child a composition according to the present invention.

Another aspect of the present invention provides a method of promoting or assisting the transition from a milk-based diet to solid food in an infant and/or a young child, the method comprising administering to the infant and/or the young child a composition according to the present invention.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

### Example 1: B. longum Transitional Microorganism

B. longum transitional strains were isolated from the feces of breast-fed infants using Eugon Tomato Agar (ETA). Obtained isolates were sequenced using PacBio to obtain a fully closed assembled genome for each of the strain. Each strain was deposited in the internal Nestle Culture Collection (NCC, Lausanne, Switzerland) and at the Collection Nationale de Microorganisms (CNCM) at the Pasteur Institute (Paris, France) together with their genome sequence data. The genome of the strains was compared by Average Nucleotide Identity (ANI) using OrthoAni (https://www.ezbiocloud.net/tools/orthoani) to other publicly available genomes representing the overall diversity of the B. longum species (Table 1), and to the Metagenomic Assembled Genomes (MAG) obtained from metagenomic sequences issued from infant feces of the same cohort.

TABLE 1
list of genomes used for ANI analysis and their publicly available
references.

| | | Nestlé | |
|---|---|---|---|
| | | Culture | |
| | | Collection | |
| | | internal | Genome |
| Taxonomy | Strain number | number | reference¹ |
| B. longum | CNCM I-5683 | NCC 5000 | Available at the |
| | | | CNCM |
| B. longum | CNCM I-5684 | NCC 5001 | Available at the |
| | | | CNCM |
| B. longum | CNCM I-5685 | NCC 5002 | Available at the |
| | | | CNCM |
| B. longum | CNCM I-5686 | NCC 5003 | Available at the |
| | | | CNCM |
| B. longum | CNCM I-5687 | NCC 5004 | Available at the |
| | | | CNCM |
| B. longum subsp. suis | BSM11-5 | NA | GCF_001870705.1 |
| B. longum | 3_mod | NA | GCF_902167615.1 |
| subsp. infantis | | | |
| B. longum subsp. | JDM301 | NA | CP002010 |
| longum | | | |
| B. longum | BXY01 | NA | GCF_000730205.1 |
| B. longum subsp. | CMCC_P0001 | NA | GCF_000410595.1 |
| longum | | | |
| B. longum subsp | SU-851 | NA | GCF_016882605.1 |
| suillum | | | |
| B. longum subsp | JCM19995 (T) | NCC 3079 | GCF_017132755 |
| suillum | | | |
| B. longum subsp. suis | 2074B | NA | GCF_016759645.1 |
| B. longum subsp. suis | Su859 (T) | NA | GCF_900103055.1 |
| B. longum subsp. suis | DSM-20211 (T) | NA | GCF_000771285.1 |
| B. longum subsp. suis | LMG_21814 (T) | NA | GCF_000741625.1 |
| B. longum subsp. suis | 209B | NA | GCF_016759725.1 |
| B. longum subsp. suis | UMA026 | NA | PHUM01000001 |
| B. longum subsp. suis | AGR2137 | NA | GCF_000421385.1 |
| B. longum subsp. | NCC 2075 | NCC 2705 | GCF_000007525.1 |
| longum | | | |
| B. longum subsp. | DJO10A | NA | GCF_000008945.1 |
| longum | | | |
| B. longum subsp. | 157F | NA | GCF_000196575.1 |
| longum | | | |
| B. longum subsp. | DSM_20219 (T) | NA | GCF_900104835.1 |
| longum | | | |
| B. longum subsp. | JCM_1217 | NA | GCF_000196555.1 |
| longum | | | |
| B. | ATCC_15697 | NA | JDTT01000001 |
| longum subsp infantis | (T) | | |
| B. | Bi-26 | NA | GCF_004919065.2 |
| longum subsp infantis | | | |
| B. | JCM_11347 | NA | CP062951 |
| longum subsp infantis | | | |

¹Genome references for NCC 5000, NCC 5001, NCC 5002, NCC 5003, NCC 5004, NCC 5005 are as given above.
(T) stands for typestrain.

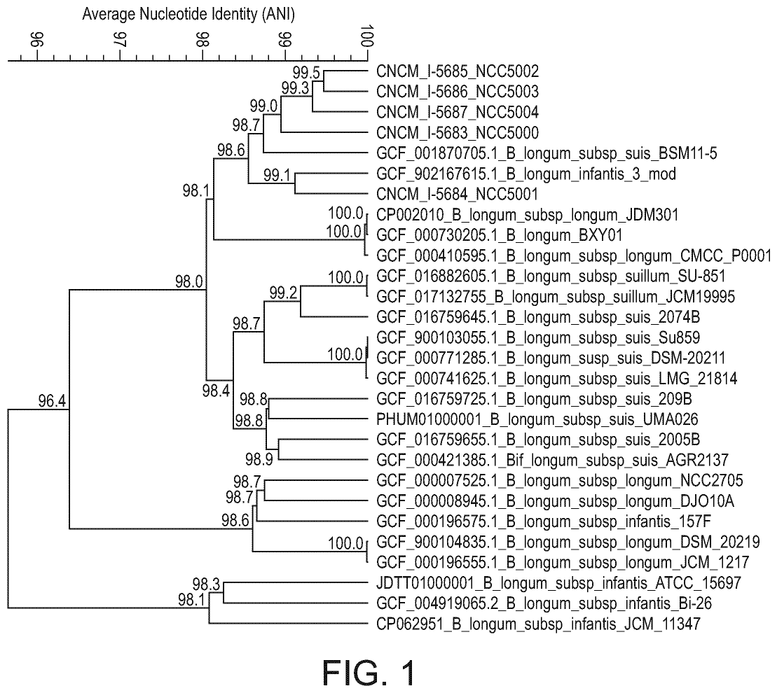

The analysis demonstrates that the newly described strains group together with the MAGs obtained from the same cohort, defining a well delineated clade belonging to the B. longum species. Two previously isolated strains BSM11-5 and 3_mod are found to be grouped within this newly described clade. The clade is genetically different from B. longum subspecies longum (96.40% ANI) subspecies. The clade is related, while still clearly distinct, to B. longum subspecies suis/suillum (98.207%), and to the group of strains (JDM301, CMCC_P0001 and BXY01) previously suggested to be a new B. longum subspecies (O'Callaghan et al. 2015), sharing an identity of 98.260% to this group of strains. FIG. 1 shows ANI UPGMA based phylogenetic tree. The scale represents the percentage (%) of identity at each branch point.

A selection of the above mentioned genomes, representing the diversity of the B. longum subspecies, were annotated for Carbohydrate-Active enZYmes(CAZY) using the dbCAN annotation pipeline (http://bcb.unl.edu/dbCAN/). Results showed that B. longum subsp. longum, B. longum subsp. Suis and B. longum subsp. suillum strains contained a GH20 (lacto-N-biosidase) enzyme, implicated in the degradation and metabolization of Lacto-N-tetraose (LNT). Similarly to B. longum subsp. infantis strains, B. longum transitional strains also possessed a similar enzyme, and in addition harbored GH29 (fucosidase) encoding genes which are implicated in the degradation and metabolization of fucosylated human milk oligo-saccharides, such as 2′FL, 3′FL or diFL. Additionally, three of the strains (CNCM 1-5684, BSM1-15& 3_mod) also harbor a GH 33 (sialidase) encoding gene implicated in the degradation and metabolization of sialilated HMO such as 3′SL or 6′SL (Table 2).

TABLE 2
Number of genes encoding for GH20 (lacto-N-biosidase), GH29 (α-
fucosidase), GH95 ((α-fucosidase/(α-galactosidase) and GH33 (sialidase)
glucohydrosylhydrase family enzymes in each of the represented genomes.

| | | | | No of | No of |
|---|---|---|---|---|---|
| | | | | predicted | predicted |
| | | No of | No of | GH95 | GH33 |
| | Genome | predicted GH20 | predicted GH29 | encoding | encoding |
| CAZYmes_pred | reference | encoding genes | encoding genes | genes | genes |
| B. longum subsp. Longum | GCF_ | 1 | 0 | 0 | 0 |
| DSM 20219 (T) | 900104835.1 | | | | |
| B. longum subsp. longum | GCF_ | 1 | 0 | 0 | 0 |
| NCC 2705 | 000007525.1 | | | | |
| B. longum subsp infantis | JDTT01000001 | 3 | 3 | 1 | 2 |
| ATCC 15697 (T) | | | | | |
| B. | Nestlé | 3 | 1 | 1 | 2 |
| longum subsp infantis LM | internal | | | | |
| G 11588 | | | | | |
| B. longum subsp. suis DS | GCF_ | 1 | 0 | 1 | 1 |
| M 20211 (T) | 000771285.1 | | | | |
| B. longum subsp. suillum | GCF_ | 1 | 0 | 1 | 0 |
| JCM 19995 (T) | 017132755 | | | | |
| B. longum spp CNCM I- | Available at | 1 | 1 | 1 | 0 |
| 5683 (NCC 5000) | the CNCM | | | | |
| B. longum spp CNCM I- | Available at | 3 | 2 | 1 | 2 |
| 5684 | the CNCM | | | | |
| (NCC 5001) | | | | | |
| B. longum spp CNCM I- | Available at | 2 | 1 | 1 | 0 |
| 5685 | the CNCM | | | | |
| (NCC 5002) | | | | | |
| B. longum spp CNCM I- | Available at | 1 | 1 | 1 | 0 |
| 5686 | the CNCM | | | | |
| (NCC 5003) | | | | | |
| B. longum spp CNCM I- | Available at | 1 | 1 | 1 | 0 |
| 5687 | the CNCM | | | | |
| (NCC 5004) | | | | | |
| B. longum spp. BSM1-15 | GCF_ | 1 | 1 | 1 | 1 |
| | 001870705.1 | | | | |
| B. longum spp. 3 mod | GCF_ | 3 | 2 | 1 | 2 |
| | 902167615.1 | | | | |
| B. longum spp. JDM301 | CP002010 | 1 | 1 | 1 | 0 |

All newly obtained genomes were compared and aligned with the genome of two strains (B. longum subsp. Infantis ATCC15697 and B. kashiwanohense DSM 21854) belonging to species for which the genes responsible for fucosylated HMOs utilization were elucidated (James et al. 2019).

### Results

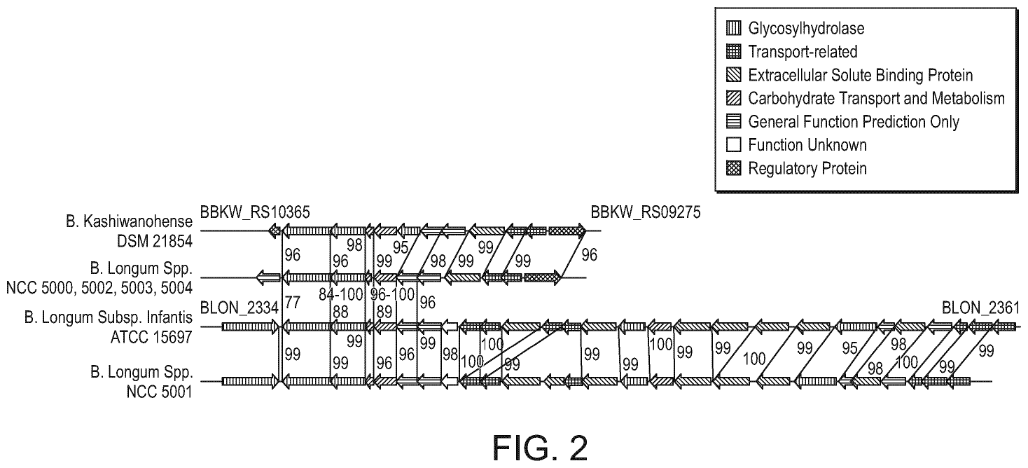

As shown in FIG. 2, all newly described strains contained genes responsible for the utilization of fucosylated HMOs. While NCC 5001 organization reflects the one of B. longum subsp. infantis ATCC 15697, all other strains (NCC 5000, NCC 5002, NCC 5003, NCC 5004) harbor a gene organization closer to that of B. kashiwanohense DSM 21854. Overall, the similarity to the well described fucosidases of B. longum subsp. infantis ATCC 15697 is above 77% (for BLON_2334) and 88% (BLON_2335) in all newly described strains.

### Example 2: Utilization of Fucosylated HMOs

All strains retrieved from the Nestle Culture Collection (Table 7) were reactivated from a freeze-dried stock, using two successive culturing steps (16 h, 37° C., anaerobiosis) in MRS supplemented with 0.05% cysteine (MRSc). Reactivated cultures were then centrifuged, washed and resuspended in 1 volume of PBS. Washed cells were used to inoculate MRS based medium without a carbon source (MRSc-C) (10 g I-1 of bacto proteose peptone n° 3, 5 g I-1 bacto yeast extract, 1 g I-1 Tween 80, 2 g I-1 di-ammonium hydrogen citrate, 5 g I-1 sodium acetate, 0.1 g I-1 magnesium sulphate, 0.05 g I-1 manganese sulfate, 2 g I-1 di-sodium phosphate, 0.5 g I-1 cysteine) in which glucose, 2′FL or 3′FL were added as unique carbon source at a concentration of 0.5%. Growth was then performed in a 96 well microplate, with a volume of 200 μl per well. Incubation was performed in anaerobiosis for 48 h, and optical density was measured in a spectrophotometer at 600 nm. As shown in FIG. 3, all B. longum transitional strains grew on fucosylated HMOS.

### Results

Surprisingly, all B. longum transitional strains grew better on 3′FL than 2′FL and reached a higher cell density on this carbohydrate. This behavior indicates a preference for 3′FL over 2′FL (ratios from 1.8 to 2.8—see FIG. 4), which is not observed in B. longum subsp. infantis LMG 11588

TABLE 3
list of the strains (and corresponding numbers) used for the individual
fucosylated HMO growth studies

| | Internationally | Nestlé Culture |
|---|---|---|
| | recognized | Collection |
| Taxonomy | deposit number | internal number |
| Bifidobacterium | LMG 11588 | NCC 3089 |
| longum subsp. infantis | | |
| Bifidobacterium longum | CNCM I-5683 | NCC 5000 |
| Bifidobacterium longum | CNCM I-5684 | NCC 5001 |
| Bifidobacterium longum | CNCM I-5685 | NCC 5002 |
| Bifidobacterium longum | CNCM I-5686 | NCC 5003 |
| Bifidobacterium longum | CNCM I-5687 | NCC 5004 |

### Example 3: Production of Short Chain Fatty Acids (SCFA)

We performed fecal batch fermentation to evaluate the capacity of specific HMOs (i.e 2′FL or 3FL) and inoculation of B. longum transitional strains to modulate SCFAs production by the complex infant microbiome. At the start of the experiment, 2′FL or 3FL were added in a concentration corresponding with approximately 2.5 g/L carbohydrates, to sugar-depleted complex medium containing the nutrients typically present in the colon. As the source of the colonic microbiota, frozen fecal sample of a healthy infant donor was defrosted and prepared for fecal inoculum and added in the tubes. Several conditions were tested i.e fermentation of HMO (2′FL or 3FL) without strain supplementation, with supplementation of B longum transitional strain NCC5002 or supplementation of B longum transitional strain NCC5004. Each fermentation occurs for 48 h at 37° C., under anaerobic conditions. Each condition was performed in triplicate. Samples were collected at the beginning (TO) and at the end of the fermentation (T48). SCFAs were measured by proton nuclear magnetic resonance (¹H-NMR) technique. The SCFA values (in arbitrary units) are derived from the integrals of the NMR peak corresponding to the SCFA.

### Results

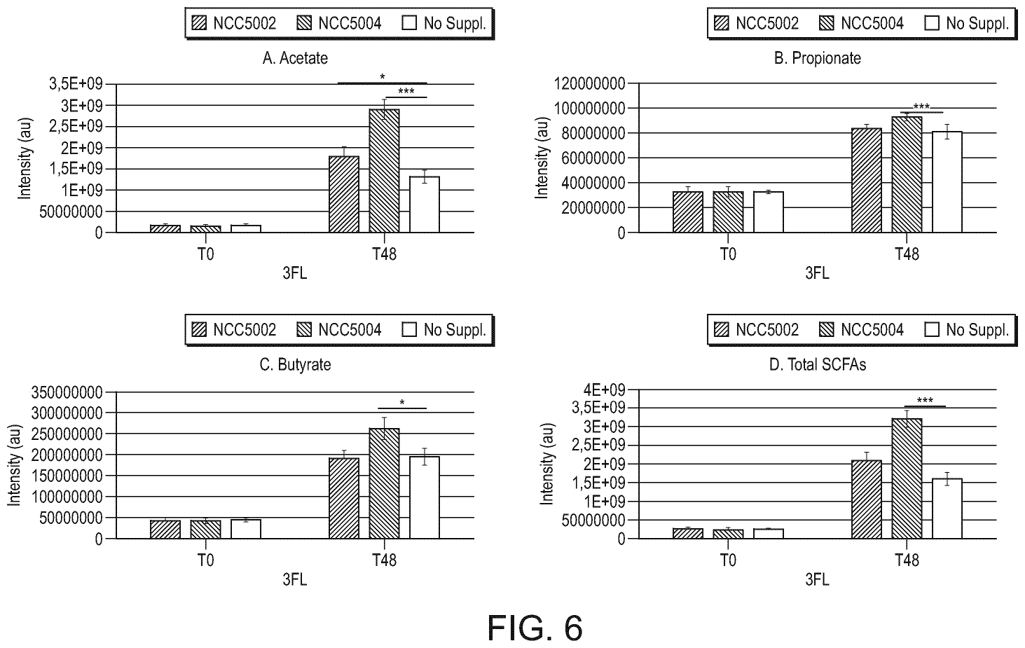

The results showed that after 48 h fermentation with 2FL, the supplementation of NCC5002 increased propionate and buyrate production while NCC5004 supplementation increased acetate and total SCFAs by comparison to a fermentation without strain supplementation (FIG. 5). After 48 h with 3FL, NCC5002 and NCC5004 supplementation increased acetate production. In addition, NCC5004 supplementation increased propionate, butyrate and total SCFAs production by comparison to a 3FL fermentation without strain supplementation (FIG. 6).

Altogether, these results indicated that supplementation of B. longum transitional strains promote SCFAs production of the infant microbiome during HMO fermentation.

1. (canceled)

2. Method according to claim 9 wherein the Bidobacterium longum microorganism has an Average Nucleotide Identity (ANI) of at least 96% with at least one Bifidobacterium longum strain selected in the group consisting of CNCM I-5683, CNCM I-5684, CNCM I-5685, CNCM I-5686 and CNCM I-5687, and any combination thereof.

3. Method according to claim 9, wherein the Bifidobacterium longum transitional microorganism comprises a glycosyl hydrolase family 95 (GH95, α-L-galactosidase; α-L-fucosidase; α-1,2-L-fucosidase) gene having at least 60% of identity with BLON 2335 gene present in Bifidobacterium longum subsp. infantis ATCC 15697 and/or a glycosyl hydrolase family 29 (GH29, α-L-fucosidase; α-1,3/1,4-L-fucosidase; α-1,2-L-fucosidase) having at least 60% identity with BLON_2336 gene present in Bifidobacterium longum subsp. infantis ATCC 15697.

4. Method according to claim 9, wherein 3-fucosyllactose (3-FL) is utilized by the Bifidobacterium longum transitional microorganism over 2′-fucosyllactose (2′-FL) in a ratio between 0.1:5.

5-7. (canceled)

8. A method of promoting the growth of a Bidobacterium longum transitional microorganism to modulate the gut microbiota of an infant and/or of a young child, the method comprising administering to the infant and/or to the young child a composition comprising a Bifidobacterium longum transitional microorganism, wherein the Bidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

9. A method of promoting/assisting the transition from a milk-based diet to solid food in an infant and/or in a young child, the method comprising administering to the infant and/or to the young child a composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

10. (canceled)

11. Method according to claim 8 wherein the composition comprises at least one prebiotic oligosaccharide selected in the group consisting of 2′-O-fucosyllactose (2FL), 3′-O-fucosyllactose (3FL), lactodifucotetraose/difucosyllactose (DFL), 3′-O-sialyllactose (3-SL), 6′-O-sialyllactose (6′-SL) and lacto-N-tetraose (LNT) and any combination thereof.

12. Method according to claim 9 wherein the composition comprises 34 wt % to 85 wt % of 2′-FL, 10 wt % to 40 wt % of LNT, 4 wt % to 14 wt % of DFL and 9 wt % to 31 wt % of 3-SL and 6′-SL combined.

13-17. (canceled)

18. A method of promoting the growth of the Biidobacterium longum transitional microorganism that preferentially utilizes 3-fucosyllactose (3-FL) over 2′-fucosyllactose (2′-FL) to modulate the gut microbiota of an infant and/or of a young child, the method comprising administering to the infant and/or to the young child a composition comprising a Bifidobacterium longum transitional microorganism, wherein the Bifidobacterium longum transitional microorganism is capable of utilizing prebiotic oligosaccharides and wherein the Bifidobacterium longum transitional microorganism preferentially utilizes 3-fucosyllactose (3-FL) over 2′-fucosyllactose (2′-FL).

19. (canceled)
